(19) United States
(12) Patent Application Publication
(10) Pub. No.: US 2011/0184159 A1
(43) Pub. Date: Jul. 28, 2011

(54) PROCESS FOR PRODUCTION OF RADIOPHARMACEUTICALS

(75) Inventors: Matthias Friebe, Berlin (DE); Keith Graham, Berlin (DE); Mathias Berndt, Berlin (DE)

(73) Assignee: Bayer Schering Pharma Aktiengesellschaft

(21) Appl. No.: 13/002,774

(22) PCT Filed: Jun. 24, 2009
(86) PCT No.: PCT/EP09/04536

§ 371 Date: Apr. 14, 2011

(30) Foreign Application Priority Data
Jul. 7, 2008 (EP) 08075611.7

(51) Int. Cl. C07H 1/00; C07C 17/361; C07C 303/30

(52) U.S. Cl. 536/18.2; 570/142; 558/53; 536/28.54

(57) ABSTRACT

The present invention relates to novel processes for the production of F-18 labeled radiotracers for Positron Emission Tomography (PET). The invention also comprises radiopharmaceutical kits using these processes.

### FIELD OF THE INVENTION

The present invention relates to novel processes for the production of F-18 labeled radiotracers for Positron Emission Tomography (PET). The invention also comprises radiopharmaceutical kits using these processes.

### BACKGROUND OF THE INVENTION

Due to its favorable half-life of 110 minutes and the low β⁺ energy (635 keV) F-18 is currently the most important isotope for Positron Emission Tomography (Wüst, F. (2005) Amino Acids, 29, 323-339.) However, the relatively short half-live requires fast processes for synthesis and purification of F-18 labeled compounds.

A common protocol for the nucleophilic production of a F-18 labeled radiotracer involves the steps of:

Production of F-18 isotope in a cyclotron by ¹⁸O (p,n)¹⁸F reaction.

Passing of the aqueous [F-18]fluoride solution through a anion exchange resin (e.g. QMA, PS-30).

Elution of [F-18]fluoride using a base/solvent mixture (commonly used: Kryptofix™ (4,7,13,16,21,24-Hexaoxa-1,10-diazabicyclo[8.8.8]-hexacosane), potassium carbonate in acetonitrile/water or tetraalkylammonium salts in acetonitrile/water).

Drying of the mixture by heating, gas stream and/or vacuum, optionally addition of acetonitrile and repeated drying.

Addition of a precursor in an organic solvent.

Nucleophilic fluorination at RT-180° C. or by microwave irradiation.

Optionally, subsequent reactions or protecting group transformations.

It is also possible to add the base/solvent mixture directly to the aqueous [F-18]fluoride solution without trapping of the [F-18]fluoride on a cartridge. However, the drying procedure is similar and might take longer in case of large volume of the aqueous [F-18]fluoride solution.

As mentioned before, due to the short half live of F-18 (110 min) fast and reliable processes for the production of F-18 radiotracers are needed. The step of removing water by azeotropic drying/evaporation demands up to 30% of the total time for the production of the labeled molecule starting from the aqueous [F-18]fluoride solution.

To separate [F-18]fluoride from target water, electrochemical methods were described, e.g.: K. Hamacher, et al.; Appl. Rad. Isot. 2002, 519-523; WO/2008/001098. Anodic deposition of [F-18]fluoride allows a separation from target water. By rinsing the electrochemical cell with an anhydrous solvent, no further azeoptropic drying step is necessary prior fluorination processes. However, special electrochemical cells are needed for those kind of protocols.

The use of ionic liquids for nucleophilic radiofluorination processes was reported (K. D. Wook et al., Nucl. Med. Biol., 2003, 345-350; WO2003076366). A typical procedure involves the steps of addition of aqueous [F-18]fluoride solution to an ionic liquid ([bmim][OTf]) and Cs₂CO₃ in H₂O at room temperature, the addition of a precursor in acetonitrile at 120° C., stirred for 8 min without capping to allow water and acetonitrile to escape from the reaction vial, cooling of the reaction, extraction using diethyl ether and purification of the crude reaction mixture by chromatography.

The problem to be solved by the invention is to provide a method, that allows a nucleophilc radiofluorination in organic solvents without azeotropic drying/evaporation prior addition of the precursor.

### DESCRIPTION OF THE INVENTION

One aspect of the present invention relates to methods for manufacturing radiofluorinated compounds, involving the steps of:

Passing aqueous [F-18]fluoride solution through a material A for trapping of [F-18]fluoride on the material A.

Optionally, drying of material A by gas stream G or by passing a solvents S through material A.

Elution of [F-18]fluoride from material A using a base/solvent mixture B.

Optionally, passing base/solvent mixture B through a material C prior and/or after passing B through material A.

Optionally, additional washing of material C with another portion of mixture B or solvent L.

Addition of precursor D.

Nucleophilic fluorination to synthesize radiofluorinated compound R—¹⁸F.

Optionally, subsequent reactions to convert R—¹⁸F to R′—¹⁸F.

Optionally, purification of R—¹⁸F or R′—¹⁸F.

Optionally, formulation of R—¹⁸F or R′—¹⁸F.

without an azeotropic drying/evaporation step prior addition of the precursor D.

A is a resin or solid, that allows trapping of [F-18]fluoride.
Optionally cartridges or columns consisting of A could be used.
In a preferred embodiment, A is an anion exchange material.
In a more preferred embodiment, A is a QMA or PS-30 cartridge.

G is a gas.
In a preferred embodiment, S is selected from the group comprising air, nitrogen, helium, argon, carbon dioxide.

S is a solvent or solvent mixture.
In a preferred embodiment, S is an anhydrous solvent or solvent mixture.
In a more preferred embodiment, S is selected from the group comprising acetonitrile, DMF, DMSO, DMAA, THF, alcohols, toluene, benzene, dichlorobenzenes, dichloromethane, xylenes, sulfolanes, and mixtures thereof.

B is a mixture of a base E and a organic solvent or a mixture of organic solvents L.
Optionally, B contains a complexing agent or a phase transfer catalyst (e.g. kryptofix, crown ether).
Optionally, B contains water.

E is an inorganic or organic base.
In a preferred embodiment, E is selected from the group comprising potassium salts, caesium salts, tetraalkylammonium salts, tetraalkylphosphonium salts.
In a more preferred embodiment, E is selected from the group comprising potassium carbonate, potassium bicarbonate, potassium oxalate, potassium sulfonates, potassium alkoxylates, potassium hydroxide, caesium carbonate, caesium bicarbonate, caesium alkoxylates tetraalkylammonium hydroxides, tetraalkylammonium bicarbonates, tetraalkylammonium halides, tetraalkylammonium sulfonates, tetraalkylphosphonium hydroxides, tetraalkylphosphonium bicarbonates, tetraalkylphosphonium halides, tetraalkylphosphonium sulfonates.
In a even more preferred embodiment, E is selected from the group comprising potassium carbonate, potassium bicarbonate, potassium oxalate, potassium mesylate, potassium tert-butylate, caesium carbonate, caesium bicarbonate, tetrabutylammonium hydroxide, tetrabutylammonium bicarbonate, tetrabutylammonium mesylate.

L is an organic solvent or mixture of organic solvents.
L is not an ionic liquid.
In a preferred embodiment, L is selected from the group comprising acetonitrile, DMF, DMSO, DMAA, THF, alcohols, toluene, benzene, dichlorobenzenes, dichloromethane, xylenes, sulfolanes, and mixtures thereof.
In a more preferred embodiment, L is is selected from the group comprising acetonitrile, DMF, DMSO, sulfolane, THF, text-butanol, amyl alcohol, DMAA or mixtures thereof.

C is a material, appropriate to remove water from solvents or solvents mixtures.
Optionally, cartridges or columns consisting of C could be used.
In a preferred embodiment, C is selected from the group comprising inorganic salts, inorganic oxides, resins, molecular sieves or mixtures thereof.
In a more preferred embodiment, C is selected from the group comprising sodium sulfate, magnesium sulfate, potassium carbonate, calcium chloride, calcium sulfate, barium oxide, magnesium oxide, calcium oxide, phosphorous oxide, potassium hydroxide, caesium carbonate, caesium hydroxide, molecular sieves or mixtures thereof.

D is a precursor for nucleophilc radiofluorination.
Optionally, D is dissolved in a solvent L, as described before.
In a preferred embodiment, D is selected from the group comprising R-Q.

Q is a leaving group, suitable for a substitution with [F-18]fluoride.
In a preferred embodiment, Q is selected from the group comprising iodide, bromide, chloride, sulfonates, trialkyl ammonium salts, nitro, aryl iodonium salts, heteroaryl iodonium salts.

R is an organic molecule.

R′ is an organic molecule.

Another aspect of the present invention relates to methods for manufacturing radiofluorinated compounds, involving the steps of:

Mixing of aqueous [F-18]fluoride solution with a base/solvent mixture B.

Passing the mixture of B and [F-18]fluoride through a material C.

Optionally, additional washing of material C with another portion of mixture B or solvent L.

Addition of precursor D.

Nucleophilic fluorination to synthesize radiofluorinated compound R—¹⁸F.

Optionally, subsequent reactions to convert R—¹⁸F to R′—¹⁸F.

Optionally, purification of R—¹⁸F or R′—¹⁸F.

Optionally, formulation of R—¹⁸F or R′—¹⁸F.

without an azeotropic drying/evaporation step prior addition of the precursor D.

B is a mixture of a base E and a organic solvent or a mixture of organic solvents L.
Optionally, B contains a complexing agent or a phase transfer catalyst (e.g. kryptofix, crown ether).
Optionally, B contains water.

E is an inorganic or organic base.
In a preferred embodiment, E is selected from the group comprising potassium salts, caesium salts, tetraalkylammonium salts, tetraalkylphosphonium salts.
In a more preferred embodiment, E is selected from the group comprising potassium carbonate, potassium bicarbonate, potassium oxalate, potassium sulfonates, potassium alkoxylates, potassium hydroxide, caesium carbonate, caesium bicarbonate, caesium alkoxylates tetraalkylammonium hydroxides, tetraalkylammonium bicarbonates, tetraalkylammonium halides, tetraalkylammonium sulfonates, tetraalkylphosphonium hydroxides, tetraalkylphosphonium bicarbonates, tetraalkylphosphonium halides, tetraalkylphosphonium sulfonates.
In a even more preferred embodiment, E is selected from the group comprising potassium carbonate, potassium bicarbonate, potassium oxalate, potassium mesylate, potassium tent-butylate, caesium carbonate, caesium bicarbonate, tetrabutylammonium hydroxide, tetrabutylammonium bicarbonate, tetrabutylammonium mesylate.

L is an organic solvent or mixture of organic solvents.
L is not an ionic liquid.
In a preferred embodiment, L is selected from the group comprising acetonitrile, DMF, DMSO, DMAA, THF, alcohols, toluene, benzene, dichlorobenzenes, dichloromethane, xylenes, sulfolanes, and mixtures thereof.
In a more preferred embodiment, L is is selected from the group comprising acetonitrile, DMF, DMSO, sulfolane, THF, tent-butanol, amyl alcohol, DMAA or mixtures thereof.

C is a material, appropriate to remove water from solvents or solvents mixtures.
Optionally, cartridges or columns consisting of C could be used.
In a preferred embodiment, C is selected from the group comprising inorganic salts, inorganic oxides, resins, molecular sieves or mixtures thereof.
In a more preferred embodiment, C is selected from the group comprising sodium sulfate, magnesium sulfate, potassium carbonate, calcium chloride, calcium sulfate, barium oxide, magnesium oxide, calcium oxide, phosphorous oxide, potassium hydroxide, caesium carbonate, caesium hydroxide, molecular sieves or mixtures thereof.

D is a precursor for nucleophilc radiofluorination.
Optionally, D is dissolved in a solvent L, as described before.
In a preferred embodiment, D is selected from the group comprising R-Q.

Q is a leaving group, suitable for a substitution with [F-18]fluoride.
In a preferred embodiment, Q is selected from the group comprising iodide, bromide, chloride, sulfonates, trialkyl ammonium salts, nitro, aryl iodonium salts, heteroaryl iodonium salts.

Furthermore, another aspect of the present invention relates to kits for carrying out a nucleophilic substitution according to the present invention.

In particular, the invention comprises:
1. A process of preparing a fluorine-18 labeled compound comprising the steps:
   a. Trapping of [F-18]fluoride on material A,
   b. Eluting the [F-18]fluoride from material A using a solution B,
   c. Addition of precursor D,
   d. Nucleophilic substitution,
whereas,
   solution B consist of an organic solvent L or a mixture of organic solvents L and a base E,
and
   solvent L is not an ionic liquid,
and
   the process comprises no azeotropic drying or evaporation after elution of [F-18]fluoride from material M prior addition of precursor D.
2. A process according to count 1, wherein a gas G and/or a solvent S is passed through material A after trapping of [F-18]fluoride and prior elution using B.
3. A process according to count 2, wherein water is removed from material A by passing G and/or S through A.
4. A process according to counts 2-3, wherein G is selected from the group comprising air, nitrogen, helium, argon, carbon dioxide, and mixtures thereof.
5. A process according to counts 2-4, wherein S is selected from the group comprising air, acetonitrile, DMF, DMSO, THF, alcohols, toluene, benzene, sulfolan, and mixtures thereof.
6. A process according to count 1-5, wherein material A is a anion exchange material or cartridge or column made of anion exchange material.
7. A process according to counts 1-6, wherein material A is a QMA or PS-30 cartridge.
8. A process according to counts 1-7, wherein L consists of mixture of acetonitrile, DMF, DMSO, sulfolane, THF, tert-butanol, amyl alcohol, DMAA or mixtures thereof.
9. A process according to counts 1-8, wherein B contains water.
10. A process according to counts 1-9, wherein solution B not contains water.
11. A process according to counts 1-10, wherein B contains a complexing agent or a phase transfer catalyst.
12. A process according to count 11, wherein B contains kryptofix or a crown ether.
13. A process according to counts 1-12, wherein base E is selected from the group comprising a:
   a. potassium salt,
   b. caesium salt,
   c. rubidium salt,
   d. tetraalkylammonium salt,
   e. tetraalkylphosphonium salt.
14. A process according to counts 1-13, wherein base E is selected from the group comprising:
   a. potassium carbonate,
   b. potassium bicarbonate,
   c. potassium oxalate,
   d. potassium sulfonates,
   e. potassium tert-alkoxylates,
   f. caesium carbonate,
   g. caesium bicarbonate
   h. tetrabutylammonium hydroxide,
   i. tetrabutylammonium bicarbonate,
   j. tetrabutylammonium mesylate.
15. A process according to counts 1-14, wherein solution B is passed through material C prior elution of [F-18]fluoride from material A, wherein material C is a material appropriate for removing water from organic solvents.
16. A process according to counts 1-15, wherein solution B is passed through material C after elution of [F-18]fluoride from material A, wherein material C is a material appropriate for removing water from organic solvents.
17. A process according to counts 15-16, wherein a drying cartridge or column consisting of material C is used.
18. A process according to counts 15-17, wherein material C is selected from the group comprising
   a. inorganic salts,
   b. inorganic oxides,
   c. resins,
   d. molecular sieves,
or mixtures thereof.
19. A process according to count 18, wherein material C is selected from the group comprising
   a. sodium sulfate,
   b. magnesium sulfate,
   c. potassium carbonate,
   d. calcium chloride,
   e. calcium sulfate,
   f. barium oxide,
   g. magnesium oxide,
   h. calcium oxide,
   i. phosphorous oxide,
   j. potassium hydroxide,
   k. caesium carbonate,
   l. caesium hydroxide,
   m. molecular sieves,
or mixtures thereof.
20. A process of preparing a fluorine-18 labeled compound comprising the steps:
   a. Mixing of aqueous [F-18]fluoride with solution B,
   b. Passing the mixture of aqueous [F-18]fluoride and B through material C.
   c. Addition of precursor D,
   d. Nucleophilic substitution,
whereas,
   solution B consist of an organic solvent L or a mixture of organic solvents L and a base E,
   optionally B also contains a complexing agent or a phase transfer catalyst,
   optionally B also contains water,
and
   solvent L is not an ionic liquid,
and
   C is a material appropriate for removing water from organic solvents,
and
   the process comprises no azeotropic drying or evaporation prior addition of precursor D.
21. A process according to count 20, wherein L consists of mixture of acetonitrile,

DMF, DMSO, sulfolane, THF, tert-butanol, amyl alcohol, DMAA or mixtures thereof.
22. A process according to counts 20-21, wherein B contains water.
23. A process according to counts 20-21, wherein solution B not contains water.
24. A process according to counts 20-23, wherein B contains a complexing agent or a phase transfer catalyst.
25. A process according to count 24, wherein B contains kryptofix or a crown ether.
26. A process according to counts 20-25, wherein base E is selected from the group comprising a:
   a. potassium salt,
   b. caesium salt,
   c. rubidium salt,
   d. tetraalkylammonium salt,
   e. tetraalkylphosphonium salt.
27. A process according to counts 20-26, wherein base E is selected from the group comprising:
   a. potassium carbonate,
   b. potassium bicarbonate,
   c. potassium oxalate,
   d. potassium sultanates,
   e. potassium tert-alkoxylates,
   f. caesium carbonate,
   g. caesium bicarbonate
   h. tetrabutylammonium hydroxide,
   i. tetrabutylammonium bicarbonate,
   j. tetrabutylammonium mesylate.
28. A process according to counts 20-27, wherein a drying cartridge or column consisting of material C is used.
29. A process according to counts 20-27, wherein material C is selected from the group comprising
   a. inorganic salts,
   b. inorganic oxides,
   c. resins,
   d. molecular sieves,
or mixtures thereof.
30. A process according to count 29, wherein material C is selected from the group comprising
   a. sodium sulfate,
   b. magnesium sulfate,
   c. potassium carbonate,
   d. calcium chloride,
   e. calcium sulfate,
   f. barium oxide,
   g. magnesium oxide,
   h. calcium oxide,
   i. phosphorous oxide,
   j. potassium hydroxide,
   k. caesium carbonate,
   l. caesium hydroxide,
   m. molecular sieves,
or mixtures thereof.

### EXAMPLES

### Example 1

### Use of Na₂SO₄ Filled Chromafix Dry Cartridge, Synthesis of 2-(2-[F-18]Fluoro-Ethyl)-Naphthalene in Acetonitrile with K₂CO₃/Kryptofix

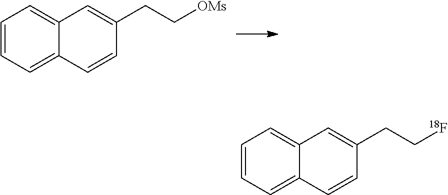

Aqueous [F-18]fluoride solution was passed through a QMA cartridge (QMA light; LOT#023336307A; waters; preconditioned with 10 mL 0.5 M K₂CO₃-solution and 10 mL water). Measured activity of the QMA cartridge: 105 MBq. 10 mL air, 10 mL acetonitrile, 10 mL air were passer through the QMA cartridge. A Chromafix Dry cartridge (Chromafix Dry S Lot# 88.071; Macherey-Nagel) was connected to the QMA cartridge. 1 mL K₂CO₃/Kryptofix-solution (1 mg K₂CO₃, 5 mg kryptofix, 950 μL acetonitrile, 50 μL water) was passed first through the chromafix dry cartridge and then through the QMA cartridge. Remaining activity on the QMA cartridge: 18 MBq, activity of the solution: 83 MBq. 5 mg Methanesulfonic acid 2-naphthalen-2-yl-ethyl ester were added to the K₂CO₃/Kryptofix[F-18]fluoride solution. The mixture was heated in a sealed vial at 100° C. for 10 min. After cooling to r.t., the conversion was determined by radio-TLC (silica gel, hexane/ethyl acetate=9/1, non-radioactive 2-(2-Fluoro-ethyl)-naphthalene was used as standard):

### Example 2

### Use of Na2SO4 Filled Chromafix Dry Cartridge, Synthesis of 2-(2-[F-18]Fluoro-Ethyl)-Naphthalene in Acetonitrile/t-BuOH with K₂CO₃/Kryptofix

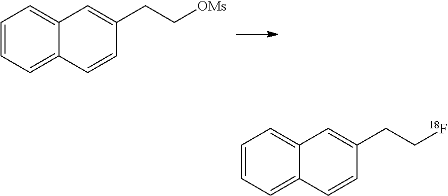

Aqueous [F-18]fluoride solution was passed through a QMA cartridge (QMA light; LOT#023336307A; waters; preconditioned with 10 mL 0.5 M K₂CO₃-solution and 10 mL water). Measured activity of the QMA cartridge: 92 MBq. 10 mL air, 10 mL acetonitrile, 10 mL air were passer through the QMA cartridge. A Chromafix Dry cartridge (Chromafix Dry S Lot# 88.071; Macherey-Nagel) was connected to the QMA cartridge. 1 mL K₂CO₃/Kryptofix-solution (1 mg K₂CO₃, 5 mg kryptofix, 750 μL t-BuOH, 200 μL acetonitrile, 50 μL water) was passed first through the chromafix dry cartridge and then through the QMA cartridge. Remaining activity on the QMA cartridge: 11 MBq, activity of the solution: 78 MBq. 5 mg Methanesulfonic acid 2-naphthalen-2-yl-ethyl ester were added to the K₂CO₃/Kryptofix[F-18]fluoride solution. The mixture was heated in a sealed vial at 100° C. for 10 min. After cooling to r.t., the conversion was determined by radio-TLC (silica gel, hexane/ethyl acetate=9/1, non-radioactive 2-(2-Fluoro-ethyl)-naphthalene was used as standard):

### Example 3

### Use of Na2SO₄ Filled Chromafix Dry Cartridge, Synthesis of 2-(2-[F-18]Fluoro-Ethyl)-Napthalene in Acetonitrile with Bu₄NOH

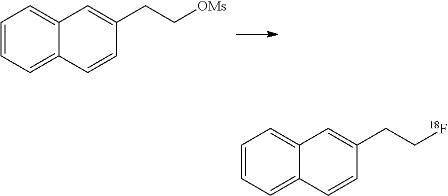

Aqueous [F-18]fluoride solution was passed through a QMA cartridge (QMA light; LOT#023336307A; waters; preconditioned with 10 mL 0.5 M K₂CO₃-solution and 10 mL water). Measured activity of the QMA cartridge: 206 MBq. 10 mL air, 10 mL acetonitrile, 10 mL air were passer through the QMA cartridge. A Chromafix Dry cartridge (Chromafix Dry S Lot# 88.071; Macherey-Nagel) was connected to the QMA cartridge. 1 mL Bu₄NOH-solution (10 μL 40% Bu₄NOH in water, 990 μL acetonitrile) was passed first through the chromafix dry cartridge and then through the QMA cartridge. Remaining activity on the QMA cartridge: 31 MBq, activity of the solution: 175 MBq. 5 mg Methanesulfonic acid 2-naphthalen-2-yl-ethyl ester were added to the K₂CO₃/Kryptofix[F-18]fluoride solution. The mixture was heated in a sealed vial at 100° C. for 10 min. After cooling to r.t., the conversion was determined by radio-TLC (silica gel, hexane/ethyl acetate=9/1, non-radioactive 2-(2-Fluoro-ethyl)-naphthalene was used as standard):

### Example 4

### Synthesis Toluene-4-Sulfonic Acid 2-[F-18]Fluoro-Ethyl Ester in Acetonitrile/t-BuOH with KOtBu

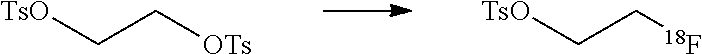

Aqueous [F-18]fluoride solution was passed through a QMA cartridge (QMA light; LOT#023336307A; waters; preconditioned with 10 mL 0.5 M K₂CO₃-solution and 10 mL water). Measured activity of the QMA cartridge: 163 MBq. 10 mL air, 10 mL acetonitrile, 10 mL air were passed through the QMA cartridge. 1 mL kryptofix/potassium tert-butylate-solution (5 mg kryptofix, 0.8 mg KOtBu in 800 μL t-BuOH, 200 μL acetonitrile) was passed through the QMA cartridge. Remaining activity on the QMA cartridge: 24 MBq, activity of the solution: 132 MBq. 5 mg ethylene ditosylate were added to the solution. The mixture was heated in a sealed vial at 100° C. for 10 min. After cooling to r.t., the conversion was determined by radio-HPLC (C 18, water/acetonitrile=100/0-5/95):

### Example 5

### Use of Na₂SO₄ Filled Cartridge, Radiolabeling of Acetyl Mannose Triflate

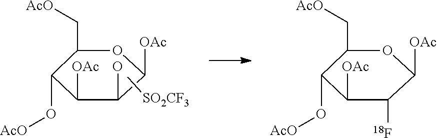

Aqueous [F-18]fluoride solution was passed through a QMA cartridge (QMA light; LOT#023336307A; waters; preconditioned with 10 mL 0.5 M K₂CO₃-solution and 10 mL water). Measured activity of the QMA cartridge: 940 MBq. 10 mL air, 10 mL acetonitrile, 10 mL air were passed through the QMA cartridge. A SepPak Dry cartridge (Waters; Cat. No.: WAT054265) was connected to the QMA cartridge. 2 mL K₂CO₃/Kryptofix-solution (1 mg K₂CO₃, 5 mg kryptofix, 950 μL cetonitrile, 50 μL water) were passed through the QMA cartridge and the dry cartridge. Remaining activity on the QMA cartridge: 62 MBq, activity of the solution: 830 MBq.

An aliquot of the solution (500 mL) was given to 5 mg mannose triflate precursor (ABX; Cat. No.: 100.1000). The mixture was heated in a sealed vial at 100° C. for 10 min. After cooling to r.t., the conversion was determined by radio-TLC (silica gel, hexane/ethyl acetate 1/1).

### Example 6

### Use of Na₂SO₄ Filled Cartridge Radiolabeling of 3-N-Boc-5′-O-Dimethoxytrityl-3′-O-Nosyl-Thymidine

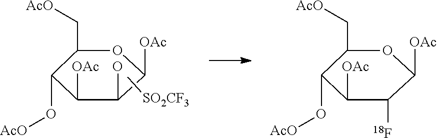

Aqueous [F-18]fluoride solution was passed through a QMA cartridge (QMA light; LOT#023336307A; waters; preconditioned with 10 mL 0.5 M K₂CO₃-solution and 10 mL water). Measured activity of the QMA cartridge: 940 MBq. 10 mL air, 10 mL acetonitrile, 10 mL air were passed through the QMA cartridge. A SepPak Dry cartridge (Waters; Cat. No.: WAT054265) was connected to the QMA cartridge. 2 mL K₂CO₃/Kryptofix-solution (1 mg K₂CO₃, 5 mg kryptofix, 950 μacetonitrile, 50 μL water) were passed through QMA cartridge and the dry cartridge. Remaining activity on the QMA cartridge: 62 MBq, activity of the solution: 830 MBq.

An aliquot of the solution (500 mL) was given to 5 mg mannose 3-N-Boc-5′-O-dimethoxytrityl-3t-O-nosyl-thymidine (ABX; Cat. No.: 1240.0040). The mixture was heated in a sealed vial at 100° C. for 10 min. After cooling to r.t., the conversion was determined by radio-TLC (silica gel, hexane/ethyl acetate 1/1).

### Example 7

### Use of Water-Free Elution Radiolabeling of Acetyl Mannose Triflate

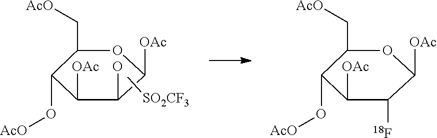

Aqueous [F-18]fluoride solution was passed through a QMA cartridge (QMA light;

LOT#023336307A; waters; preconditioned with 10 mL 0.5 M K₂CO₃-solution and 10 mL water). Measured activity of the QMA cartridge: 470 MBq. 10 mL air, 10 mL acetonitrile, 10 mL air were passed through the QMA cartridge. 2 mL K₂CO₃/Kryptofix-solution (1.6 mg potassium tert-butylate, 5 mg kryptofix, 200 μL acetonitrile, 800 μL tert-butanol) were passed through QMA cartridge. Remaining activity on the QMA cartridge: 106 MBq, activity of the solution: 355 MBq.

An aliquot of the solution (500 mL) was given to 5 mg mannose triflate precursor (ABX; Cat. No.: 100.1000). The mixture was heated in a sealed vial at 100° C. for 10 min. After cooling to r.t., the conversion was determined by radio-TLC (silica gel, hexane/ethyl acetate 1/1).

### Example 8

### Use of Water-Free Elution, Radiolabeling of 3-N-Boc-5′-O-dimethoxytrityl-3′-O-Nosyl-Thymidine

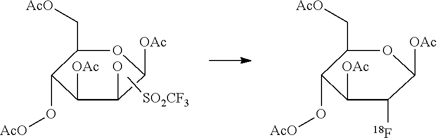

Aqueous [F-18]fluoride solution was passed through a QMA cartridge (QMA light; LOT#023336307A; waters; preconditioned with 10 mL 0.5 M K₂CO₃-solution and 10 mL water). Measured activity of the QMA cartridge: 989 MBq. 10 mL air, 10 mL acetonitrile, 10 mL air were passed through the QMA cartridge. 2 mL K₂CO₃/Kryptofix-solution (1.6 mg potassium tert-butylate, 5 mg kryptofix, 200 μL acetonitrile, 800 μL tert-butanol) were passed through the QMA cartridge. Remaining activity on the QMA cartridge: 208 MBq, activity of the solution: 778 MBq.

An aliquot of the solution (500 mL) was given to 5 mg mannose 3-N-Boc-5′-O-dimethoxytrityl-3′-O-nosyl-thymidine (ABX; Cat. No.: 1240.0040). The mixture was heated in a sealed vial at 100° C. for 10 min. After cooling to r.t., the conversion was determined by radio-TLC (silica gel, hexane/ethyl acetate 1/1).

1. A process of preparing a fluorine-18 labeled compound comprising the steps:
a. Trapping of [F-18]fluoride on material A,
b. Eluting the [F-18]fluoride from material A using a solution B,
c. Addition of precursor D,
d. Nucleophilic substitution,
whereas,
solution B consist of an organic solvent L or a mixture of organic solvents L and a base E,
and
solvent L is not an ionic liquid,
and
the process comprises no azeotropic drying or evaporation after elution of [F-18]fluoride from material M prior addition of precursor D.

2. A process according to claim 1, wherein a gas G and/or a solvent S is passed through material A after trapping of [F-18]fluoride and prior elution using B.

3. A process according to claim 2, wherein water is removed from material A by passing G and/or S through A.

4. A process according to claims 2-3, wherein G is selected from the group comprising air, nitrogen, helium, argon, carbon dioxide, and mixtures thereof

5. A process according to claims 2-4, wherein S is selected from the group comprising air, acetonitrile, DMF, DMSO, THF, alcohols, toluene, benzene, sulfolan, and mixtures thereof

6. A process according to claim 1-5, wherein material A is a anion exchange material or cartridge or column made of anion exchange material.

7. A process according to claims 1-6, wherein material A is a QMA or PS-30 cartridge.

8. A process according to claims 1-7, wherein L consists of mixture of acetonitrile, DMF, DMSO, sulfolane, THF, tert-butanol, amyl alcohol, DMAA or mixtures thereof.

9. A process according to claims 1-8, wherein B contains water.

10. A process according to claims 1-8, wherein solution B not contains water.

11. A process according to claims 1-10, wherein B contains a complexing agent or a phase transfer catalyst.

12. A process according to claims 11, wherein B contains kryptofix or a crown ether.

13. A process according to claims 1-12, wherein base E is selected from the group comprising a:
a. potassium salt,
b. caesium salt,
c. rubidium salt,
d. tetraalkylammonium salt,
e. tetraalkylphosphonium salt.

14. A process according to claims 1-13, wherein base E is selected from the group comprising:
a. potassium carbonate,
b. potassium bicarbonate,
c. potassium oxalate,
d. potassium sulfonates,
e. potassium tert-alkoxylates,
f. caesium carbonate,
g. caesium bicarbonate
h. tetrabutylammonium hydroxide,
i. tetrabutylammonium bicarbonate,
j. tetrabutylammonium mesylate.

15. A process according to claims 1-14, wherein solution B is passed through material C prior elution of [F-18]fluoride from material A, wherein material C is a material appropriate for removing water from organic solvents.

16. A process according to claims 1-15, wherein solution B is passed through material C after elution of [F-18]fluoride from material A, wherein material C is a material appropriate for removing water from organic solvents.

17. A process according to claims 15-16, wherein a drying cartridge or column consisting of material C is used.

18. A process according to claims 15-17, wherein material C is selected from the group comprising
a. inorganic salts,
b. inorganic oxides,
c. resins,
d. molecular sieves,
or mixtures thereof.

19. A process according to claim 18, wherein material C is selected from the group comprising
a. sodium sulfate,
b. magnesium sulfate,
c. potassium carbonate,
d. calcium chloride,
e. calcium sulfate,
f. barium oxide,
g. magnesium oxide,
h. calcium oxide,
i. phosphorous oxide,
j. potassium hydroxide,
k. caesium carbonate,
l. caesium hydroxide,
m. molecular sieves,
or mixtures thereof.

20. A process of preparing a fluorine-18 labeled compound comprising the steps:
a. Mixing of aqueous [F-18]fluoride with solution B,
b. Passing the mixture of aqueous [F-18]fluoride and B through material C.
c. Addition of precursor D,
d. Nucleophilic substitution,
whereas,
solution B consist of an organic solvent L or a mixture of organic solvents L and a base E,
optionally B also contains a complexing agent or a phase transfer catalyst,
optionally B also contains water,
and
solvent L is not an ionic liquid,
and
C is a material appropriate for removing water from organic solvents,
and
the process comprises no azeotropic drying or evaporation prior addition of precursor D.

21. A process according to claim 20, wherein L consists of mixture of acetonitrile, DMF, DMSO, sulfolane, THF, tert-butanol, amyl alcohol, DMAA or mixtures thereof.

22. A process according to claims 20-21, wherein B contains water.

23. A process according to claims 20-21, wherein solution B not contains water.

24. A process according to claims 20-23, wherein B contains a complexing agent or a phase transfer catalyst.

25. A process according to claims 24, wherein B contains kryptofix or a crown ether.

26. A process according to claims 20-25, wherein base E is selected from the group comprising a:
a. potassium salt,
b. caesium salt,
c. rubidium salt,
d. tetraalkylammonium salt,
e. tetraalkylphosphonium salt.

27. A process according to claims 20-26, wherein base E is selected from the group comprising:
a. potassium carbonate,
b. potassium bicarbonate,
c. potassium oxalate,
d. potassium sulfonates,
e. potassium tert-alkoxylates,
f. caesium carbonate,
g. caesium bicarbonate
h. tetrabutylammonium hydroxide,
i. tetrabutylammonium bicarbonate,
j. tetrabutylammonium mesylate.

28. A process according to claims 20-27, wherein solution B is passed through material C prior elution of [F-18]fluoride from material A, wherein material C is a material appropriate for removing water from organic solvents.

29. A process according to claims 20-28, wherein solution B is passed through material C after elution of [F-18]fluoride from material A, wherein material C is a material appropriate for removing water from organic solvents.

30. A process according to claims 28-29, wherein a drying cartridge or column consisting of material C is used.

31. A process according to claims 28-30, wherein material C is selected from the group comprising
a. inorganic salts,
b. inorganic oxides,
c. resins,
d. molecular sieves,
or mixtures thereof.

32. A process according to claim 31, wherein material C is selected from the group comprising
a. sodium sulfate,
b. magnesium sulfate,
c. potassium carbonate,
d. calcium chloride,
e. calcium sulfate,
f. barium oxide,
g. magnesium oxide,
h. calcium oxide,
i. phosphorous oxide,
j. potassium hydroxide,
k. caesium carbonate,
l. caesium hydroxide,
m. molecular sieves,
or mixtures thereof.
